(19) United States
(12) Patent Application Publication
(10) Pub. No.: US 2019/0009005 A1
(43) Pub. Date: Jan. 10, 2019

(54) Coated Medical Device and Method of Coating a Medical Device to Reduce Fibrosis and Capsule Formation

(71) Applicant: Ecole Polytechnique Federale de Lausanne (EPFL), Lausanne (CH)

(72) Inventors: Hicham Majd, Renens (CH); Giorgio Pietramaggiori, Lausanne (CH); Boris Hinz, Toronto (CA)

(21) Appl. No.: 16/040,322

(22) Filed: Jul. 19, 2018

Related U.S. Application Data
(62) Division of application No. 13/062,679, filed on Jun. 1, 2011, now abandoned.

(30) Foreign Application Priority Data
Sep. 5, 2008 (EP) 08163816.5

(51) Int. Cl. A61L 27/50; A61L 27/34; C08L 89/00

(52) U.S. Cl. CPC A61L 27/50; Y10T 428/24479; C08L 89/00; A61L 27/34; Y10T 428/24562

(57) ABSTRACT

Coating for a medical device comprising cell-adhesive proteins having the ability to reduce fibrous reaction, said coating being in the form of separate islets having an individual area which is less than 12 μm² and wherein the distance between the islets is less than 50 μm.

### CROSS-REFERENCE TO RELATED APPLICATIONS

This application is a divisional of U.S. application Ser. No. 13/062,679 filed Jun. 1, 2011, which is a US National Phase Application of PCT Patent Application No. PCT/IB2009/053897 filed Sep. 7, 2009, which claims priority to EP Patent Application No. 08163816.5, filed Sep. 5, 2008.

### FIELD OF THE INVENTION

The present invention relates to a coating technology modulating cell adhesion on the surface of medical devices. The coating prevents capsule formation and decreases fibrosis induced by foreign bodies such as medical devices.

### BACKGROUND OF THE INVENTION

Medical devices in contact with living cells and tissues induce a foreign body reaction due to their physical and chemical properties. This process results in a capsule formation around the implant that often contracts and results in malfunctioning of the device and clinically relevant complications such as pain and dysmorphism of the patient that ultimately require additional surgical operations.

The reaction of cells to a foreign material such as for example a silicone breast and aesthetic implant, a gastric band, a dental implant, an orthopedic implant, hearth heart valve, etc., which cannot be phagocytated, enzymatically digested or otherwise eliminated, is the formation of fibrotic capsule around it.

It has been reported by Prantl L., and al. (Clinical and morphological conditions in capsular contracture formed around silicone breast implants, Plast Reconstr Surg 2007, 120:275-284) that in up to 20-30% of implanted prosthesis develop capsule formation. The capsule formation is excessive and leads to clinically relevant, painful contractures around the device. Patients with excessive capsular formation need to receive a second operation to remove the capsule around the device which considerable increases in the morbidity and costs. This process significantly shortens the lifespan of the device.

This capsule is the result of a chronic inflammatory process around the material that resolves only after the formation of the capsule itself. Myofibroblasts play a main role in capsule formation and contraction in healing tissues. The capsule itself is deposited and contracted by myofibroblasts. In the wound healing process, these highly active cells proliferate and growth to occupy tissue defects and replaces them with a scar. Around the implant, myofibroblast deposit collagen fibers around the foreign material and eventually contract it. This reaction produces capsular contraction.

The problem of capsule formation has also been discussed in patent literature. In U.S. Pat. No. 4,772,285 a collagen coated soft tissue prosthesis is described for reducing capsule formation. While this patent proposes a strategy to decrease capsule formation, no mechanism is provided to effect myofibroblast formation. Further the clinical evidence is lacking. U.S. Pat. Nos. 4,955,907; 4,731,081; 5,571,183; 5,207,709; 5,354,338; 4,428,082 and 4,298,998 also propose solutions to avoid or diminish capsule formation.

All these documents have several common denominators which have the potential of making them unsuitable for resolving this problem in human beings.

For example U.S. Pat. No. 4,298,998 disclose causing a capsule to form at a predetermined, controlled distance from the surface of the implant, thus resulting in the same capsule but at a different location. The end result clinically appears to be a hard capsule for the patient and not resolving the problem.

Similarly, the implant described in U.S. Pat. No. 5,207,709 includes a plurality of fine projections extending from the outer surface arrayed in a basket weave-like, herringbone-like, or other suitable pattern to create a sinuous path for collagen formation around the implanted device. It appears that this implant actually creates or invites collagen formation again in another location around the implant and again therefore is not resolving the problem.

Still other patents relate to the implant being surrounded by a medical grade elastomer or as described in U.S. Pat. No. 4,944,749 a viscous gel coating with the membranes constructed of a suitable material such as medical grade silicone rubber, which does not react with human tissue. The outer membrane contains an amount of viscous gel, for example a silicone rubber gel of medical grade silicone.

It appears in the end that this patent still has a silicone tissue interface that has accounted for problems.

U.S. Pat. No. 4,610,690 is directed to an implant with a lubricious layer of an acrylamide polymer radiation bonded to at least one wall surface of a silicone shell or bag. Potential long-term effects in human beings of an acrylamide polymer interface are not discussed.

All these aforementioned patents continue to have unnatural chemicals as interface with human tissue, which is exactly what patients do not want in their body and what usually causes problems.

In U.S. Pat. No. 4,995,882 an organic fill solution is proposed to solve the problem. The implant is proposed the use a triglyceride fill substance such as peanut oil or sunflower seed oil as a filling substance.

Although some implants of this kind were implanted in Europe, they were never authorized for implantation in the United States and were subsequently taken off the market worldwide because of various problems.

Although many strategies have been developed to reduce capsule formation, from modifications of the surface of the implants to chemical coatings, only mixed results have been reported and, with the increase in the demand for implantable devices, the clinical problem is on the rise.

### SUMMARY OF THE INVENTION

It is therefore an object of the invention to provide a coating of a medical device and a method to produce it with which the fibrosis and capsule contraction can be further diminished or even avoided.

This can be achieved by a medical device wherein a surface of the medical device is coated with cell-adhesive proteins deposited in a matrix of islets with specific size, area and distribution, object of this invention.

This coated surface, which is supposed to come in contact with living cells and tissues to modulate cell adhesion is composed from two specific regions:

1. Area (islets) where cell (tissue) can specifically attach (coated part), and

2. Other region where cell (tissue) cannot specifically attach (non coated part).

This invention relates in general to a method and device for guiding cellular adhesion on medical devices. The invention relates to a method comprising protein islets to coat devices (implantable and external) in contact with any cell and tissue of the body.

Specifically, the invention contemplates the use of such technique in combination with devices implanted or externally applied to the body.

This proposal outlines a novel strategy based on islands of proteins to decrease the formation of fibrosis reaction and contraction around medical devices.

According to a preferred embodiment of the invention the medical device comprises silicone as a material of the medical device.

Other preferred embodiments include the use of plastic, metal and/or collagen as a material of the medical device.

According to one embodiment of the invention the medical device according to the invention comprises islets of proteins with uniform geometric shapes.

Another possibility would be the use of different geometric shapes.

Generally, to reduce the myofibroblast differentiation and fibrosis, the area of the single islets is less than 12 μm².

The topography of islets that best reduces fibrosis is composed of single islets, wherein the islets have preferably a length that is <6 μm, a width that is <2 μm and distance between them that is <6 μm.

In a preferred embodiment of the invention the medical device further comprises an additional substrate to facilitate the transfer of the islets of proteins.

The substrate which is preferably applied to the medical device before coating same with proteins could be silicone, plastic, bio resistant materials, etc.

Further the invention describes a method of coating a medical device, wherein the medical device is coated with cell-adhesive proteins in form of single islets.

As already mentioned it could be in some embodiments of advantage if the medical device is coated by a substrate before being coated by the proteins.

According to one embodiment of the invention the proteins are applied to the medical device by a stencil or mask (a template with holes of the size and distribution of the islets).

For applying the proteins according to another embodiment the stencil or mask could be brought in contact with the surface of the medical device and the proteins are transferred to the surface through holes of the stencil.

The fixing of the proteins to the medical device could be done by every possibility known from the art. As an example, it should be mentioned covalent binding, electro deposition or precipitation of proteins on the medical device and/or the substrate.

The medical device as described can be used for example for breast implants, tissue expanders, inflatable bumps, implants, transplants, prosthesis, insulin pumps, drug delivery systems, cardiovascular devices, skin substitutes, wound dressings and/or tubes.

These and other objects, features, aspects and advantages of the present invention will become more apparent from the following detailed description of the present invention when taken in conjunction with the accompanying drawings.

### BRIEF DESCRIPTION OF THE DRAWINGS

FIG. 1 shows a medical device before and after being coated according to one embodiment of the invention; and

FIG. 2 shows examples of islet shapes according to a preferred embodiment of the invention.

FIG. 3 The deposited proteins of the coating are organized in islets of a length that is <6 μm, a width that is <2 μm and distance between them that is <6 μm. The fibroblasts attach to the surface and do not become myofibroblasts.

FIG. 4 On the left, fibroblasts become myofibroblasts (white cells) on control surfaces without coating. On the right, islets f a length that is <6 μm, a width that is <2 μm and distance between them that is <6 μm impaired the formation of myofibroblasts.

FIG. 5 In the presence of a coating with islets >6 μm in length, a width >2 μm and distance between them >6 μm, fibroblasts attach and become myofibroblasts (white cells).

FIG. 6 Control implants, on the left, induce fibrosis (in black, myofibroblasts) and capsular contraction at the interface with living tissues. The coating organized in islets of proteins of a length that is <6 μm, a width that is <2 μm and distance between them that is <6 μm reduces this reaction several folds.

### DETAILED DESCRIPTION OF THE INVENTION

The invention describes a new method of micro-deposition of cell-adhesive proteins and molecules onto the surface of medical devices, such as, but not limited to highly deformable and elastic substrates (such as, but not limited to silicone), Titanium, Plastic, Polyurethane and generally all materials used for medical devices and implants.

The micro-deposited islets of proteins guide cell adhesion on medical devices with the objective to reduce the fibrotic reaction of the cells and tissues in contact with the device.

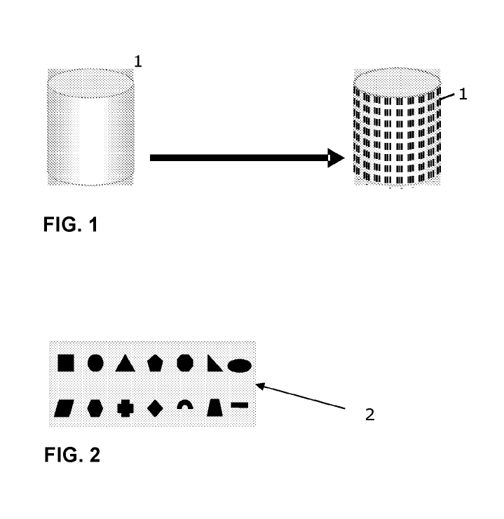

As shown in FIG. 1 according to an embodiment of the invention a microperforated stencil or mask is used to transfer proteins to coat a medical device. 1. Deposition of the proteins and molecules is in the form of specific islets. 2. Protein islets include any possible geometrical shape and spatial organization and individual area. Protein islets (2) are deposited to modulate cell and tissue adhesion to medical devices (1).

The distance between the islets may vary from 1 to 50 μm. The area of the single islets being less than 12 μm².

The distribution of protein islets according to a preferred embodiment of the invention to best reduce fibrosis includes single islets, wherein the islets have a length that is <6 μm, a width that is <2 μm and distance between them that is <6 μm.

In another embodiment, deposited islets could have any geometrical shape. Examples to which the invention is not limited are illustrated in FIG. 2.

The mask or a stencil to transfer the islets is micro fabricated using technology as for example photolithography, dry and wet etching, laser cutting.

As type of mask or stencil should be mentioned soft stencils made from silicone and flexible polymers and hard stencils from silicon, hard polymer and metal.

To deposit proteins onto medical device as for example an implant surface, the stencil is first brought in conformal contact with the surface and then the proteins are transferred on the surface through the micro-holes of the stencil by covalent binding using 3-aminopropyltriethoxysilane (APTES) and glutaraldehyde or formaldehyde. After the crosslinking of proteins, the stencil is removed and the pattern remains on the implant surface.

Another possible method is electro deposition or precipitation of proteins on implant or medical device surface through the micro-holes of stencil or mask. These possibilities are just examples and should not limit the invention to this approach of deposition.

Our results in vitro and in vivo illustrate the importance of the size of the islets in reducing fibrosis and contraction around implants proving the relevance of this invention. We found that the main mechanism responsible for limiting the differentiation of human dermal fibroblasts in myofibroblasts (the main cell responsible for fibrosis and contraction) is the area of adhesion.

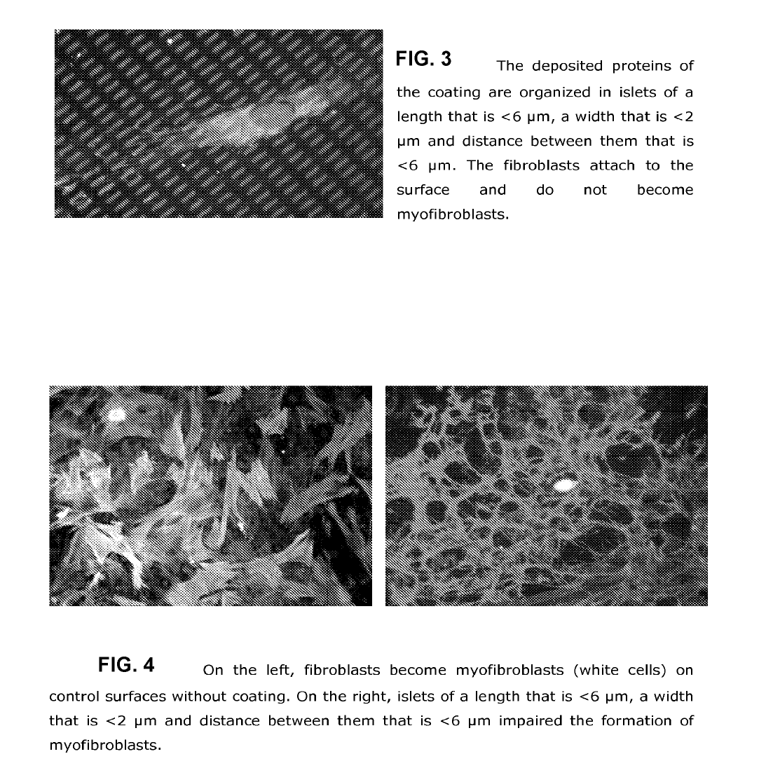

In our experiments, the islets represent areas of adhesion (focal adhesions) for the cells and dimensions of the islets of a length that is <6 μm, a width that is <2 μm and distance between them that is <6 μm impaired the formation of myofibroblasts (FIG. 3).

The specific islet size and distribution with a length <6 μm, a width <2 μm and distance between them <6 μm does not allow fibroblasts to exert forces on the surface where they attach and become myofibroblasts (the main cell responsible for fibrosis and contraction).

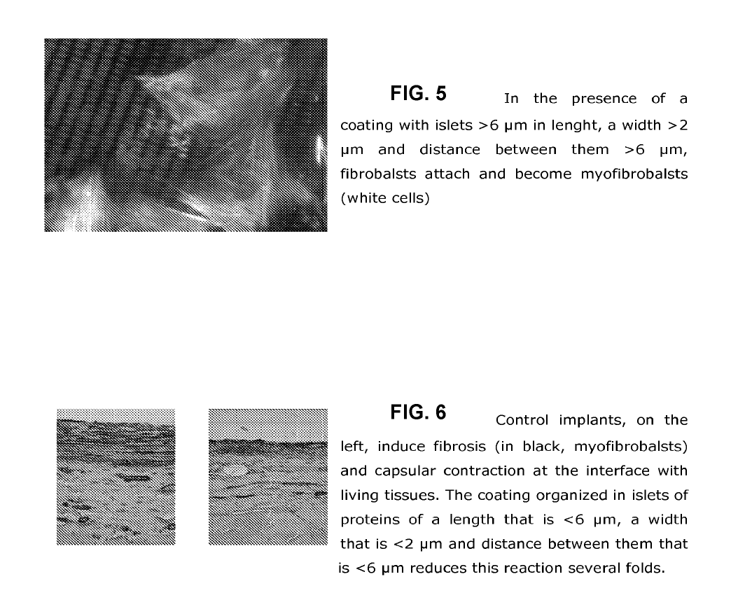

In vitro, we showed that this specific size and distribution of proteins reduced 10-fold the differentiation of human dermal fibroblasts to myofibroblasts compared to devices coated with other size and distribution of proteins (FIG. 4). The deposition of islets of higher size allowed the differentiation of fibroblasts in myofibroblasts. These later cells were seen in high percentage (up to 20%) when the surface was coated with larger islets than the specific ones provided by this invention (FIG. 5).

In vivo, silicone pads (1×1 cm) covalently coated with a stencil or mask, as described in the methods, on both side with protein islets with a length <6 μm, a width <2 μm and distance between them <6 μm, were inserted in subcutaneous pockets, in female Wistar rats (250-350 g). On the scapular region of the dorsum of these animals, 4 coated silicone pads were placed. Each animal received four implants: two implants coated with islets with a length <6 μm, a width <2 μm and distance between them <6 μm; and two non coated silicone implants, alternating the location on successive animals. Results at 6 months show a 3-fold decrease in capsule formation around implants coated with islets of proteins with a length <6 μm, a width <2 μm and distance between them <6 μm (FIG. 6).

In vitro and in vivo results show that the transformation of fibroblasts into myofibroblasts is crucial in the development of fibrosis and capsule

contraction around medical implants and underline the importance of this invention specifically limiting this event.

The optimized protein islets deposition with e.g. a length <6 μm, a width <2 μm and distance between them <6 μm can be applied to any medical device such as, but not limited to:
Silicone breast implants
Tissue expanders and inflatable pumps
Bone, and cartilage and orthopaedic implants
Tendon, nerve, and ligament transplants, substitutes and implants
Implantable pumps, such as insuline pumps and drug delivery systems
Cardiovascular devices, such as pace makers, vascular prosthesis, heart valves, vascular stents
Skin substitutes and cell scaffolds
Wound dressings, including dressings and wound interfaces connected to a vacuum (negative pressure dressings)
Acoustic waves (such as shockwave therapy)
Ear, throat, nose and eye implants
Brain, central and peripheral nervous system implants and prosthesis
Tubes, connecting tubes, drainage tube systems

1. A medical device comprising a covalently attached coating, the coating comprising cell-adhesive proteins having the ability to reduce fibrous reaction,
wherein the cell-adhesive proteins comprise collagen, fibronectin, vitronectin, or combinations of fibronectin and vitronetic,
wherein the coating is in the form of separate islets each having an individual area that is less than 12 μm², and
wherein the distance between the islets is between 1 μm and 50 μm.

2. The medical device of claim 1, wherein the distance between the islets is less than 6 μm.

3. The medical device of claim 1, wherein the islets have a uniform geometrical shape.

4. The medical device of claim 1, wherein the islets have a non-uniform geometrical shape.

5. The medical device according to claim, wherein the islets have a length that is less than 6 μm, a width that is less than 2 μm and a distance between islets that is less than 6 μm.
